(19) United States
(12) Patent Application Publication
(10) Pub. No.: US 2019/0321412 A1
(43) Pub. Date: Oct. 24, 2019

(54) METHODS AND COMPOSITIONS USING DEHYDRATED HUMAN AMNION/CHORION MEMBRANE ALLOGRAFT AND AMNIONIC MEMBRANE ALLOGRAFT SUSPENSIONS FOR THE TREATMENT OF HAIR LOSS

(71) Applicant: Stephen Edward Mahoney, Houston, TX (US)

(72) Inventor: Stephen Edward Mahoney, Houston, TX (US)

(21) Appl. No.: 16/392,584

(22) Filed: Apr. 23, 2019

Related U.S. Application Data
(60) Provisional application No. 62/661,393, filed on Apr. 23, 2018.

(51) Int. Cl. A61K 35/50; A61K 35/16; A61K 35/19; A61K 9/00; A61P 17/14

(52) U.S. Cl. CPC A61K 35/50; A61K 35/16; A61P 17/14; A61K 9/0019; A61K 35/19

(57) ABSTRACT

The present invention relates to compositions and methods for the treatment of hair loss and for promoting hair growth by subcutaneously or intradermally administering injections of compositions comprising therapeutically-effective amounts of extracts of one or more dehydrated human amnion/chorion membrane allografts (“dHACM”) and/or amnionic membrane allografts (“AMA”) suspensions, alone or in combination with one or more extracts of platelet rich plasma (“PRP”), wherein the dHACM and/or AMA suspensions are administered in therapeutic dosages and in a composition for use in the disclosed method.

### CROSS REFERENCE TO RELATED APPLICATIONS

This application claims priority under 35 U.S.C. § 119 to U.S. Provisional Application Ser. No. 62/661,393, filed on Apr. 23, 2018, the disclosure of which is hereby expressly incorporated by reference in its entirety.

### BACKGROUND OF DISCLOSURE

### Field of the Invention

The present invention relates to compositions and methods for the treatment of male and female hair loss and promoting hair growth, particularly androgenetic alopecia.

### Background of the Invention

Non-scarring male and female hair loss, clinically referred to as androgenetic alopecia (“AGA”), is a condition which affects approximately 50% of both men and women over the course of their lifetime. AGA is a progressive alopecia resulting in a decrease in the density and thickness of the hair, miniaturization of the hair follicle and potentially significant baldness. Androgenetic alopecia results in the gradual thinning beginning at the vertex or crown of the scalp to the forehead. By the age of 50 approximately 85% of men have significantly thinning hair and 20-53% of women show thinning often beginning with symptoms around the age of 21. Men commonly show temporal recession and vertex involvement which can progress to total baldness. Women have similar pattern areas of thinning but rarely progress to complete baldness.

Other types of hair loss in males and females include telogen effluvium (“TE”), alopecia areata (“AA”), traumatic alopecia, central centrifugal cicatricial alopecia (“CCCA”), frontal fibrosing alopecia (“FFA”), and lichen planopilaris (“LPP”). Scarring alopecias such as CCCA, FFA and LPP are extremely difficult to treat because the hair follicles are replaced by fibrosis and scarring making the hair loss permanent and frequently unresponsive to previous compositions and procedures.

### DESCRIPTION OF THE RELATED ART

A number of previous compositions and procedures that have claimed to be able to treat AGA, include topical MINOXIDIL® (Rogains), oral FINASTERIDE® (Propecia)/spironolactone and hair transplant surgery. These previous drug treatments have been only marginally effective, as well as expensive and intensive in use, and frequently have many adverse effects. In addition to being expensive, surgical hair restoration can be very painful and invasive—many times producing results that are not cosmetically appealing and scarring. Hair transplant surgery can be difficult in patients with severe hair loss or scarring because obtaining enough donor hair follicles is not possible or the follicles are not able to grow in the scarred transplant areas.

The prior art is replete with topical “hair-loss” compositions containing a plethora of active ingredients, many purporting to be naturally-derived from botanical sources (e.g., antioxidants, hydroxy acids, retinoids and short chain peptide derivatives) and claiming to help reduce the appearance of signs of androgenetic alopecia.

Platelet Rich Plasma (PRP)

The use of platelet rich plasma (“PRP”) therapy has spanned multiple fields of medicine including orthopedics, pain management, wound healing and most recently, in the current invention—dermatological therapy for hair loss. PRP which is derived from the patient's own blood contains more than 30 bioactive proteins and 7 growth factors which have important roles in tissue healing and stem cell activation and neovascularization. These growth factors include Platelet derived growth factor (“PDGF”), Transforming growth factor-beta (“TGF-beta”), Epidermal growth factor (“EGF”), Vascular endothelial growth factor (“VEGF”), Fibroblast growth factor (“FGF”), Connective tissue growth factor (“CTGF”), Insulin like growth factor-1 (“IGF-1”). PRP contains approximately 800 other bioactive molecules secreted by platelets including cell adhesion molecules, cytokines, antimicrobial peptides and anti-inflammatory molecules.

The US Food and Drug Administration (“FDA”) considers PRP a blood product so its oversight falls to the FDA's Center for Biologics Evaluation and Research (“CBER”) which is responsible for regulating human cells, tissues, and cellular/tissue-based products. PRP preparation systems or kits are commercially available, but those currently marketed have only received limited FDA clearance, most of which is 510(k) clearance (where a device is given clearance because it is considered equivalent to a previous device) for producing PRP mixed with bone graft materials for orthopedic uses. The FDA does not approve, through its clearance program, a kit for a particular treatment for any particular clinical indication. FDA clearance only applies to its intended use in a particular setting. For example, in some of its past warning letters, the FDA has required certain manufacturers of PRP preparation systems to add language to its label stating that the PRP prepared by the preparation system has not been evaluated for any clinical indication.

Any use of PRP outside of and FDA cleared setting, for example in an injection in a physician's office” is considered to be “off label use.” “Off label use,” however, is not prohibited by the FDA's current regulation regime if conducted by a licensed physician with the intent to practice medicine. According to FDA guidance, clinicians who use a product like a PRP kit off-label “have the responsibility to be well informed about the product, to base its use on firm scientific rationale and on sound medical evidence, and to maintain records of the product's use and effects.” The guidance further states that when the intent to practice medicine is present, the submission of an investigational new drug application (“IND”), investigational device exemption (“IDE”), or oversight from a medical board, is not required. The use of PRP kits by physicians in an office setting for treatment of hair loss would be considered “off-label” use.

Several recent studies have demonstrated the positive effects of PRP on promoting hair growth as demonstrated through increasing hair number and hair thickness. The exact mechanism of how PRP impacts the hair growth cycle is undetermined. It is theorized that platelets and the associated growth factors become activated when they are injected into the scalp and then support stem cell migration, promote blood vessel growth, and stimulate protein and collagen synthesis—all of which are important in promoting new hair growth. The platelets then help to activate and regenerate hair growth within the follicles, prolong anagen phase and increase cycling of the hair cycle from telogen (resting) to anagen (growth) phase.

Based on this literature including prior art patents, PRP injections and injecting PRP combined with various substances (e.g., combining PRP with certain proportions of basic fibroblast growth factor (bFGF) such as disclosed in U.S. Pat. Nos. 9,173,921 and 9,782,483 to Lim), have been used for treating hair loss. However, it appears the results of some injection methods vary widely, and results may be conflicting and questionable. The existing injection methods of PRP treatment, while restoring hair to some individuals, have provided inconsistent results for others suffering from hair loss.

By combining PRP with either dHACM or AMA, the present invention provides more rapid hair growth and reduction of hair loss than use of PRP by itself or with prior art substances and an achieves an apparent prolongation of the positive clinical results. As such, it has been found that the administration of PRP mixed with dHACM or AMA in combination with an intradermal or subcutaneous injection procedure step, provides a more effective method for the treatment of male and female hair loss.

dHACM and AMA

Human-derived placental tissues have been shown in randomized clinical trials to be effective for healing chronic wounds, and also have demonstrated the ability to recruit stem cells to a would site. See Massee M., et al. 2016, Dehydrated human amnion/chorion membrane regulates stem cell activity in vitro. J Biomed Mater Res Part B 2016: 1048: 1495-1503.

dHACM is a unique, thin, collagenous membrane derived from the placenta, the area in which the human fetus grows and develops within the mother's uterus. Human amniotic membrane consists of collagen layers including basement membrane and stromal matrix. Amniotic membrane is the innermost layer of the placenta consisting of a thick basement membrane and an avascular stromal matrix. It can be used as a graft and as a dressing to facilitate tissue reconstruction and to promote healing. Amniotic membranes are a robust source of pluripotent/stem cells, growth factors, highly organized collagen, matrix proteins, and anti-inflammatory cytokines. Amniotic membrane can be processed to produce dHACM. AMA is (typically in liquid form) a non-immunogenic, antibacterial, enhancer of the wound healing process, reducers of inflammation and scar formation, and recruiter of stem cells to wound sites.

dHACM and AMA have been used for a variety of reconstructive surgical procedures since the early 1990s and have promising applications in medicine, plastic surgery, orthopedics and dermatology.

The novel combination of PRP with dHACM or AMA disclosed by the present invention has created a “super-charged PRP” mixture that has yielded improved and faster results for stimulation of hair growth and reduction of hair loss over PRP alone. According to applicant's experience, injections with PRP mixed with dHACM or AMA as disclosed in the present invention provides substantial areas of hair regrowth in as fast as one month and after only one or two injection sessions. Additional applications of this dHACM or AMA and PRP combination therapy such as micro-needling with this mixture for treatment of various modes of hair loss is also being studied, as are both stand-alone dHACM and AMA suspensions. Varying the dosages of these suspensions may prove important in initiation and maintenance of hair growth.

There remains a need for new and improved methods for reducing hair loss requiring fewer treatments. This need is met by the methods and compositions of the present invention.

### SUMMARY OF THE INVENTION

The present invention relates to the inventor's novel discovery of treating hair loss and stimulating hair growth by administering dHACM suspensions and/or AMA suspensions, alone or in combination with one or more extracts of PRP by subcutaneously or intradermally injecting such compositions into a patient's scalp.

In addition to treating AGA, the mixture and processes in the present disclosure may be used for treating various hair loss disorders including, but not limited to, alopecia greata, telogen effluvium, anagen effluvium, cicatricial, alopecia, scarring alopecia, scalp thinning; hair shaft abnormalities such as trichorrexis nodosa, loose anagen syndrome, trichotillomania, and traction alopecia; infectious hair disorders such as tiniea capitis, sebohorreic dermatitis, and follicullitus of the scalp; genetic disorders such as androgenic alopecia, and patients undergoing hair loss due to chemotherapy, radiation therapy, hormonal imbalance (e.g., thyroid conditions such as hypothyroidism and hyperthyroidism, pregnancy, child birth, discontinuation of birth control pills and changes in menstrual cycle), fungal infection of the scalp such as ringworm, medicines causing hair loss such as anti-coagulants, medicine for gout, depression, high blood pressure, and certain heart medications. The inventive composition may also be used to treat hair loss related to environmental factors and chemicals. The mixture of the present disclosure may also be used to treat hair loss related to other diseases such as diabetes, lupus, and poor nutrition, mental/physical stress such as due to surgery, illness, and high fever. The mixture of the present disclosure may comprise a pharmaceutically acceptable carrier.

Another object of the present invention is directed to treating hair loss and stimulating hair growth on other areas of a patient's body in addition to the scalp.

When PRP is mixed with dHACM or AMA suspensions under the present inventions process, the resulting composition has a surprising beneficial effect on increasing the number hair follicles and the density of hair growth as well as decreasing hair loss.

### BRIEF DESCRIPTION OF THE DRAWINGS

The advantages and objects of the illustrative, non-limiting embodiments will become apparent by describing them in detail along with reference to the attached drawings:

FIG. 1 shows and illustrative depiction of the non-limited treatment method for the invention in accordance with the disclosure below.

FIG. 2A shows an illustrative embodiment of a patient's target area before the treatment in accordance with the disclosure below;

FIG. 2B shows an illustrative embodiment of a target area after the treatment in accordance with the disclosure below;

FIG. 3A shows an illustrative embodiment of a patient's target area before the treatment in accordance with the disclosure below;

FIG. 3B shows an illustrative embodiment of a target area after the treatment in accordance with the disclosure below;

FIG. 4A shows an illustrative embodiment of a patient's target area before the treatment in accordance with the disclosure below;

FIG. 4B shows an illustrative embodiment of a target area after the treatment in accordance with the disclosure below;

FIG. 5A shows an illustrative embodiment of a patient's target area before the treatment in accordance with the disclosure below;

FIG. 5B shows an illustrative embodiment of a target area after the treatment in accordance with the disclosure below.

FIG. 6 shows an illustrative embodiment of a PRP mixture preparation kit in accordance with the disclosure below.

### DETAILED DESCRIPTION OF EMBODIMENTS OF THE INVENTION

The descriptions below of non-limiting embodiments of the invention disclose specific ingredients and methods, however, these embodiments are merely examples, and merely used to describe such embodiments and provide overall understanding to one skilled in the art. Accordingly, a person skilled in the art will readily recognize that the disclosure is not limited to the specific embodiments described below. For the sake of clarity and brevity some descriptions of various ingredients and processes that would be known to a person skilled in the art are omitted.

According to an exemplary embodiment, dHACM or AMA or their mixture with PRP may be administered in a therapeutically effective amount by injection intradermally or subcutaneously to treat male and female hair loss and promote hair growth. The injections are performed using a sterilized micro-needling procedure which effectively stimulates and supplies the mixture for hair growth in treated areas, such as the scalp.

In one preferred embodiment of this aspect of the invention, the dHACM or AMA is donated human tissue intended for homologous use and is available through third-party processors of human amniotic tissue, such as Synergy Biologics® and PURION® processed dHACM or AMA (MiMedx®⁾). Other commercially available dHACM or AMA could be used or processed to obtain similar results as disclosed below. All dHACM or AMA used for the present invention meets stringent specifications during donor screening and laboratory testing to reduce the risk of transmitting infectious disease. dHACM and AMA are processed from human tissue according to the American Association of Tissue Banks (AATB) standards, and is regulated as a human cell, tissue, or cellular or tissue-based product (HCT/P) under Section 361 of the Public Health Service Act. AMA or dHACM may be used as currently available from a third-party processor or supplier, or mixed with a suspending agent or other agent.

Growth Factors and Cytokines known to be used in the dHACM used in one embodiment of the present invention are:

| | |
|---|---|
| Angiogenin | Granulocyte Colony-Stimulating |
| | Factor |
| Angiopoietin-2 | Growth Differentiation Factor 15 |
| Basic Fibroblast Growth | Interferon Gamma |
| Factor | |
| Bone Morphogenetic Protein 5 | Interleukin 1 Alpha |
| Brain-Derived neurotrophic | Interleukin 1 Beta |
| Factor | |
| Endocrine Gland-Derived | Interleukin 1 Receptor Antagonist |
| Vascular Endothelial Growth | |
| Factor | |
| Epidermal Growth Factor | Interleukin 4 |
| Fibroblast Growth Factor 4 | Interleukin 5 |
| Keratinocyte Growth Factor | Interleukin 6 |
| Growth Hormone | Interleukin 7 |
| Heparin Binding EGF-Like | Interleukin 10 |
| Growth Factor | |
| Hepatocyte Growth Factor | Interleukin 12 p40 |
| Insulin-Like Growth Factor 1 | Interleukin 12 p70 |
| Insulin Like Growth Factor | Interleukin 15 |
| Binding Protein 1 | |
| Insulin Like Growth Factor | Interleukin 17 |
| Binding Protein 2 | |
| Insulin Like Growth Factor | Macrophage Colony-Stimulating |
| Binding Protein 3 | Factor |
| Insulin Like Growth Factor | Osteoprotegerin |
| Binding Protein 4 | |
| Insulin Like Growth Factor | B Lymphocyte Chemoattractant |
| Binding Protein 6 | (CXCL13) |
| Beta Nerve Growth Factor | Eotaxin 2 |
| Placental Growth Factor | Chemokine Ligand 1 (CCL1) |
| Platelet-Derived Growth | Interleukin 8 |
| Factor AA | |
| Platelet-Derived Growth | Interleukin 16 |
| Factor BB | |
| Transforming Growth Factor | Monocyte Chemotactic Protein 1 |
| Alpha | (CCL2) |
| Transforming Growth Factor | Monokine Induced by Gamma |
| Beta 1 | Interferon (CXCL9) |
| Vascular Endothelial Growth | Macrophage Inflammatory Protein 1 |
| Factor | Alpha (CCL3) |
| Tissue Inhibitor of | Macrophage Inflammatory Protein 1 |
| Metalloproteinase 1 | Beta (CCL4) |
| Tissue Inhibitor of | Macrophage Inflammatory Protein 1D |
| Metalloproteinase 2 | (MIP-5, CCL15) |
| Tissue Inhibitor of | Regulated on Activation, Normal T- |
| Metalloproteinase 4 | cell Expressed and Secreted (CCL5) |

Without wishing to be bound by a theory, applicant believes that growth factor rich medium from pluripotent AMA show promise in enhancing tissue healing and regeneration and can be further enhanced by growth factors/stem cell activators found in PRP. An advantage of dHACM and AMA allografts is their ability to promote scar-free healing. Transforming growth factor beta (TGF-b) is a soluble factor known among clinicians to induce fibrotic responses through activation of fibroblasts. AMA, in turn, is believed to inhibit the expression of TGF-b thereby reducing scar formation. Hyaluronic acid in the mesenchymal portion of amniotic membranes is thought to be the primary inhibitor of TGF-b. Research has found that TGF-b is critical in inducing regression of the hair growth cycle and thus causing anagen (growing) phase follicles to shift to telogen (resting) phase follicles. Further research has shown that epithelial cell death is induced through TGF-b activation and thus leads to more elimination or regression of hair follicles and less hair production or growth. Applicant believes these processes are fundamental to the initiation and progression of AGA in both male and female patients.

The hair growth and regression cycle are thought to be a completely self-contained process within the hair follicle stem cell niche. The decision-making mechanisms for the hair growth cycle are extrinsic and controlled by the release of these growth factors or cytokines. Altering these cytokines/growth factors within the hair follicle stem cell niche with the introduction of the invention's AMA/PRP mixture provides a novel and effective treatment for AGA and scarring alopecias which should revolutionize the future of hair growth treatments and the prevention of scarring and hair loss.

The predominant effect of TGF-b on cell proliferation is thought to be inhibition. Increased production of TGF-b is believed to induce an accumulation or increase in P27 protein (cyclic dependent kinase inhibitor). Increased estrogen is believed to cause a protein kinase degradation of P27; whereas progesterone induces a marked increase in P27. This interaction suggests that P27 and TGF-b may be central targets for growth regulation and hair loss in AGA. Increased testosterone/androgens or decreased estrogens may also play a role in AGA through this mechanism.

Without wishing to be bound by a theory, the applicant believes the unique properties of the invention's dHACM or AMA and PRP mixture inhibit the actions of TGF-b and P27 which is primary in stopping the progressive hair loss that is seen in AGA. It is thought that by keeping the hair follicles in the anagen (active growth) phase and preventing the progression to telogen (inactive or resting) phase, patients will retain more hair and stop the continued hair loss. Applicant believes that dHACM or AMA and PRP growth factors stimulate mesenchymal stem cells to regulate immune responses and control inflammation. Activation of these mesenchymal stem cells within the hair follicle is thought to cause a migration downward of the germ cell progeny toward the dermal papillae. This action appears to trigger a regeneration of the hair growth cycle turning telogen follicles into anagen follicles. Accordingly, applicant believes that stimulation by both dHACM and AMA and PRP activated growth factors are not only highly therapeutic in causing regrowth or regeneration of the hair cycle, but also in stopping the progression to the telogen phase and hair loss which is characteristic of AGA and chronic telogen effluvium. By also limiting fibroblast activity and other inflammatory cytokines, the invention's dHACM or AMA and PRP mixture controls the scarring process which further restricts hair growth and causes permanent hair loss in patients. These benefits of the present invention have been noted in improvements in minimizing scarring and increasing hair growth in patients with scarring alopecias like CCCA, FFA and LPP.

The mixture according to one embodiment of the procedure for treating hair loss is not limited, as long as it contains dHACM or AMA and PRP, and is administered subcutaneously or intradermally by injection.

The therapeutically effective amount of dHACM and/or AMA for the invention is not limited, but in preferred embodiments can be administered therapeutic amounts in a range of 25 mg to 100 mg. In another exemplary embodiment, PRP may be used in therapeutic amounts in a range of 12-18 ml to promote hair growth. These substances may be administered sequentially or as a mixture.

The amount of PRP collected from the blood of each patient for the inventive procedure varies from patient to patient, given varying composition of platelets in human blood.

The frequency of administration is not limited to a particular duration. In one embodiment, it is preferred to administer the mixture periodically for a certain time period, for example, once every 4 to 6 weeks. It is advised that the frequency of the injections be based on the age, health, condition of the patient's hair and results desired. The applicant's inventive mixture's potentiating the action of mesenchymal stem cells may also increase the therapeutic duration of the invention's treatment in a manner such that treatments can be more temporally spaced.

### Dosage

As will be appreciated by those of skill in the art, the amount(s) of ingredients (dHACM or AMA and PRP) which constitute a therapeutically-effective dose will vary based on the age, condition of the patient, extent of hair loss, other skin and health conditions (past and present), and route(s) of administration. Suitable dosing regimens can be readily selected by those skilled in the art, taking into consideration these factors.

### Non-Limiting Illustrative Treatment Method

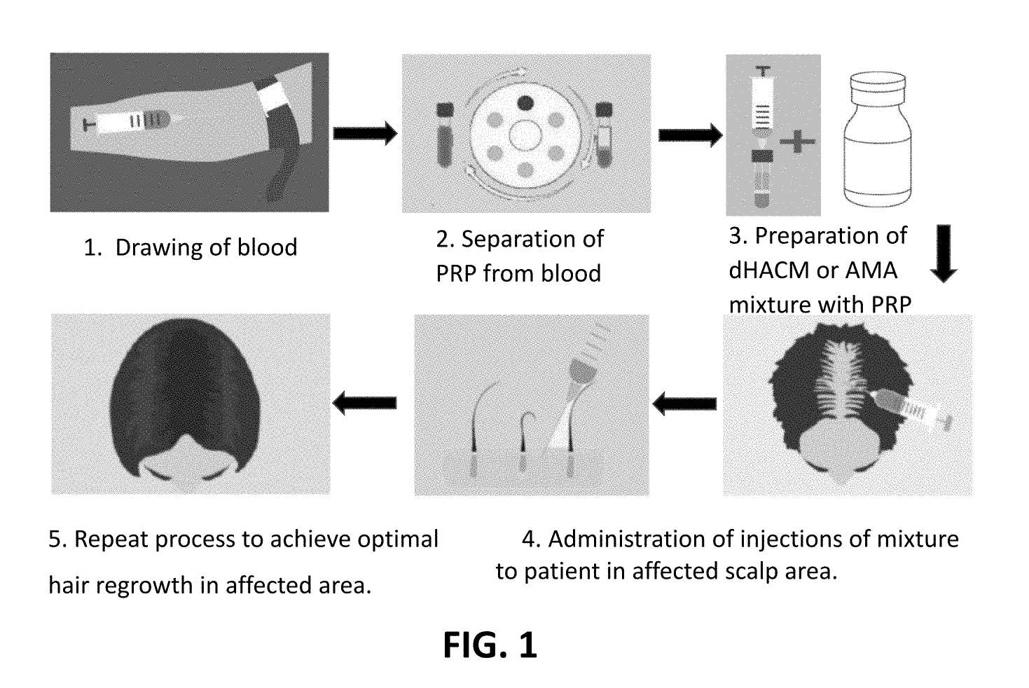

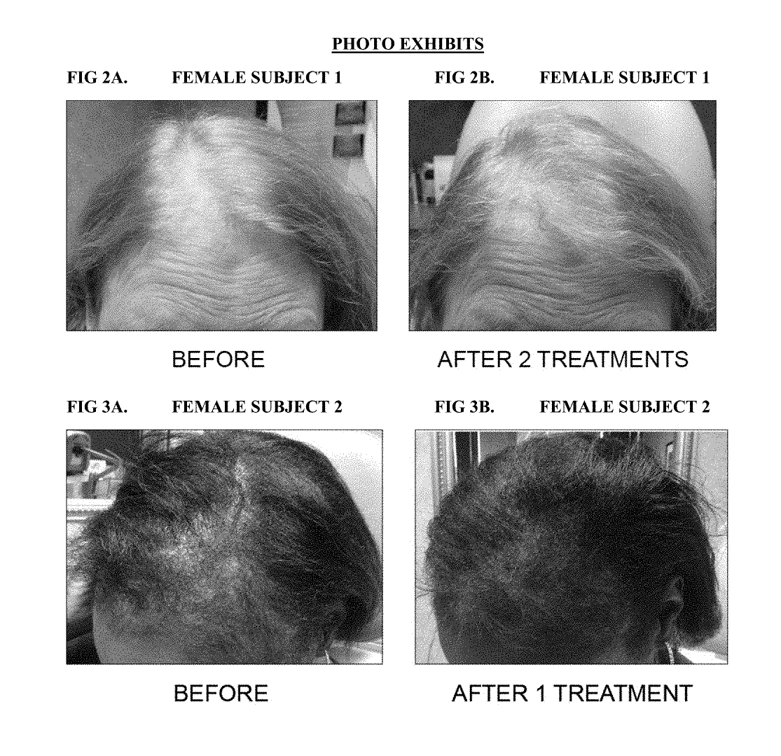

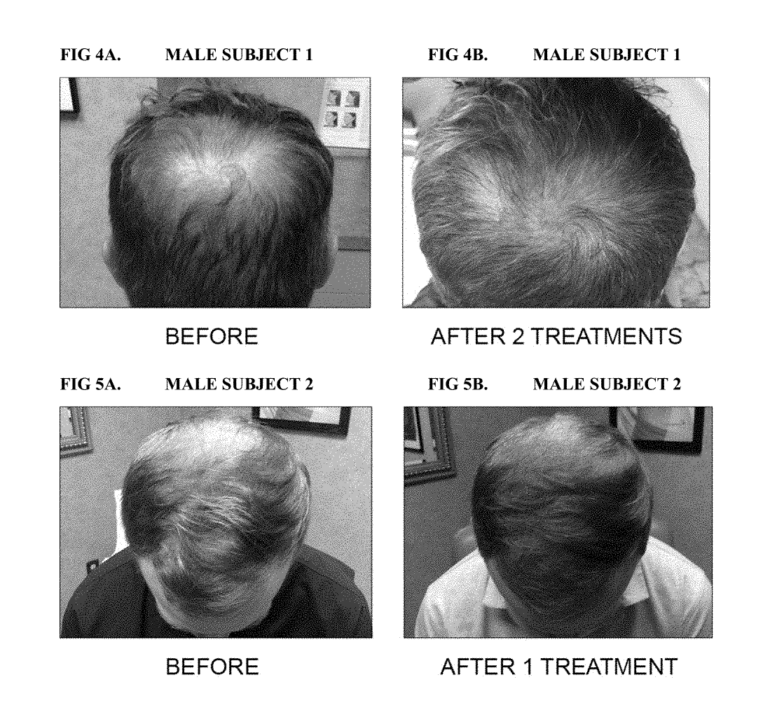

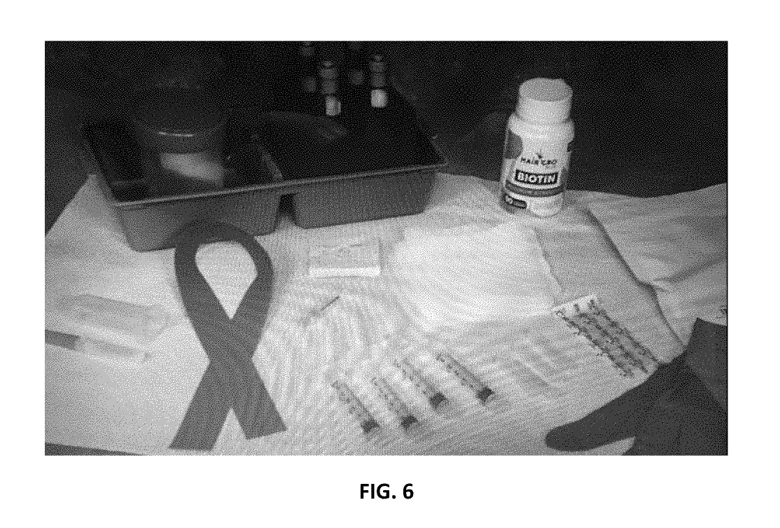

The following explanation of a non-limiting, illustrative embodiment of a treatment procedure involves the following steps as further illustrated in FIG. 1. One or more steps may be omitted or carried out in a different sequence as long as the mixture which contains dHACM or AMA, optionally in combination with PRP is intradermally or subcutaneously injected into a patient.

1. Drawing of Blood

The method of extraction of PRP in the present invention involves the drawing of whole blood from the patient using conventional methods well known in the art. The proper amount of whole blood to extract may be determined by the patient's health and the condition and extent of the area where the injections will be applied. As stated, the source of the whole blood may come from other than the patient, or a commercially available PRP product could be used. The whole blood may be appropriately stored under known methods for subsequent treatments or drawn prior to treatments. 8.5 ml tubes draw 6-8 cc's 4 of them milliliters (mls) of whole blood. Get 12-18 of PRP.

In one preferred embodiment the extraction of an amount of blood commonly drawn to harvest PRP for the subsequent injections is 20-32 ml. of whole blood to fill a plurality of collection tubes.

Following know aseptic venipuncture techniques familiar to a person of ordinary skill in clinical procedures, collecting a plurality of tubes of whole blood from the patient, filling a plurality of the sterile specimen collection tubes. Sterile techniques well known to a person of ordinary skill in clinical procedures are used at every stage of PRP collection, preparation and application to maintain a sterile environment and to reduce the risk of infection.

The blood draw occurs with the addition of an anticoagulant, such as ACD-A (acidic citrate dextrose A) or others, which prevents platelet activation prior to PRP use. Each collection tube is manually inverted a number of times in a careful manner familiar to a person of ordinary skill in clinical procedures to ensure proper mixing of the tube additive with the patient's blood.

In one embodiment, the invention is directed to use of a PRP collection, mixing and injection kit for use in practicing the invention's method in treating hair loss comprising one or more of the following: a plurality of thin gauge needles (the higher the gauge, the thinner the needle, for example, a 30 gauge needle is thinner than a 29 gauge needle); a plurality of luer lock syringes, one or more pairs of sterile gloves, one or more sterile specimen containers, a plurality of blood specimen collection tubes, one or more foam tube holders; one or more alcohol preparation pads, one or more bandages, one or more tourniquets. In addition, the kit may contain one or more dHACM or AMA suspension vials in a therapeutic dosage amount.

An exemplary embodiment of one such a PRP collection, mixing and injection kit, similar to the one illustrated in EX. 6, is used to prepare the present inventive composition for injection. In one alternative embodiment, such kit includes the following:

| | |
|---|---|
| Four (4) 18 gauge needles | Four (4) 3 ml luer lock |
| | syringes |
| Four (4) 30 gauge needles | Alcohol prep pad |
| One (1) pair sterile gloves | One (1) fabric bandage |
| One (1) sterile 4 oz | Tourniquet |
| specimen container | |
| Four (4) blood specimen | One Dehydrated |
| collection tubes | DHACM or AMA |
| | suspension vial in a |
| | therapeutic dosage |
| | amount. |
| One (1) foam tube holder | |

2. Separation of PRP from Blood.

In one preferred embodiment, the whole blood extracted in Step 1 is separated in a centrifuge and the PRP serum is extracted. The collection tubes are placed in a centrifuge in a manner familiar to a person of ordinary skill in clinical procedures. In one representative embodiment, the centrifuge is set to one spin cycle of 15 minutes. The range of speed—not divulged. In such representative embodiment, the goal of centrifugation is to achieve a platelet concentration of 1.0-1.5 million platelets per milliliter (ml.) or roughly 4-6 times the platelet concentration seen in whole blood. Once the centrifugation is complete, the each of the tubes is removed from the centrifuge. At that point, the collected blood specimen will be stratified into generally three distinct layers because of differences in specific gravity. The lower layer generally consists of red blood cells, the middle layer generally consists of white blood cells and the upper layer generally consists of PRP (which has a golden to yellowish color). The upper layer (PRP) will usually yield 15-18 ml of PRP depending on the baseline platelet count of the patient.

Following the centrifugation process, place each tube back into a foam tube holder found in the exemplary kit, making sure that the foam holder is resting flat on the surface of the counter/table.

3. Preparation of the DHACM or AMA+PRP Mixture

To enhance the clinical response, dHACM or AMA suspension is added to the PRP extract. The dHACM or AMA in sterile vials generally can be stored prior to usage up to five years in ambient conditions.

In one embodiment, the PRP mixture injection preparation technique includes:
Removing the top from the dHACM or AMA suspension vial. Wipe injection port with isopropyl alcohol prep pad.
Draw the PRP produced from step 2 from each collection tube and transfer it into a sterile mixture cup. Drawing PRP on each tube—3-4 draw up and stick in sterile cup. If suspension just inject Cup—either take 2 mls out and draw into powder—then have 19-20 ccs put that into separate syringes and inject to patient. Don't—determines what added to mixing cup. Drawing all of it out an putting in mixing cup. We landed on 25 or 40. Sterile cup
Using a syringe (in one exemplary embodiment a 3 ml syringe with 18-gauge needle), draw up approximately 2 ml of PRP from the 4 oz. sterile mixture cup (i.e. specimen container). This PRP will be used for rehydration of the dHACM when it is used. It is usually not necessary to rehydrate AMA because it is in solution. Sterile cup—put in central cup, 2 ml and remix in cup. In some cases, no rehydration step may be necessary so the dHACM or AMA product may be removed directly from the vial and added to the patient's PRP. Alternatively, the dHACM or AMA used in the present disclosure may be formulated into an appropriate formulation following a common method. The formulation may be a solid formulation such as powder and granule but for more efficiency for preventing and treating hair loss, it may be preferably formulated into a liquid agent for injection such as a solution agent, emulsion, or a suspension agent or the like.
Transfer the 2 ml PRP into the vial of dehydrated dHACM. Mix the particulate with the syringe plunger to create a full suspension of dHACM and PRP mixture in the syringe. Because the reconstituted material is viscous, use proper pre-injection techniques known in the art to reduce possible air introduction.
Using the same needle and syringe, transfer all of the reconstituted dHACM material into a sterile mixing cup. If using AMA, use a syringe to transfer all of the AMA into the sterile mixing cup.
Transfer all remaining PRP from each collection tube to the sterile mixing cup that contains the reconstituted dHACM or AMA. Once all PRP is in the specimen container along with the reconstituted dHACM or AMA, swirl the liquid a few times.
Once mixed, draw up the dHACM or AMA and PRP mixture into each of the plurality of exemplary 3 ml syringes to full 3 ml capacity. DO NOT discard any remaining PRP.
Carefully replace the exemplary 18-gauge needles with 30-gauge needles, making sure to follow OSHA'S Bloodborne Pathogens Standard for changing and disposing of needles. The PRP/AMA mixture is now ready to be injected into the patient.

4. Administration of Injections to a Patient

The mixture is injected intradermally or subcutaneously into the scalp or other target area using very small needles (e.g. the exemplary 30-gauge needle syringes) at the level of the hair follicles.

The injection treatment typically takes 15-20 minutes. A topical anesthetic can be applied to the areas prior to the injections to make the process nearly painless. Following the treatment session, the scalp may feel slightly numb but these symptoms are very temporary. No special post care treatment or down time is required. In the preferred embodiment:
The mixture should be injected intradermally or subcutaneously into the scalp areas of thinning hair and the surrounding region. Injections should be given at a depth of 2-4 mm to deliver the mixture to the depth of the dermal papillae and bulge area of the hair follicle to allow for activation of mesenchymal stem cells within the hair follicle complex.
If necessary, the clinician should be sure to part the patient's hair as the process goes along, to ensure that all areas of thinning are injected.
As the clinician injects (0.3-0.4) ml to each injection site, a cobblestone-like appearance of the skin may develop as it rises up slightly as the mixture is injected. The total amount of mixture typically corresponds to 12.0-18.0 mls.
Occasionally, there will be some minor erythema and bleeding, which will go away with slight pressure.
A “cobblestone” effect may be present, but the mixture will be reabsorbed typically in 10-15 minutes. If the mixture is injected intradermally, there is minimal discomfort and no long-term complications.
It is important to use all of the mixture. The clinician should dispose of any remaining materials in accordance with biohazardous, sharps and OSHA Bloodborne Pathogens Standard protocols.

5. Repeating the Process

In one or more embodiments, the full process of hair regrowth and decreased thinning using the applicant's inventive composition and procedure takes multiple injection sessions (typically greater than 3) and continued maintenance treatments to achieve the patient's desired results. Preferably, treatments are spaced apart every 4-6 weeks. Depending on the amount of initial hair loss, several injection sessions (2-3) may be required and occasional maintenance treatments at 6-month intervals also may be required to insure optimal hair restoration.

Applicant's results were measured qualitatively by high definition photographs taken pre-treatment, and at months 1, 2, 3, 6 and 1-year post-treatment.

### EXAMPLES

The following examples and related figures are further illustrative of the present invention. The components and specific ingredients are presented as being typical, and various modifications can be derived in view of the foregoing disclosure within the scope of the invention.

To date, the applicant's inventive treatment has been used by applicant on more than 200 patients ranging from one to fifteen treatment sessions over a three to twelve-month period. The patient's ages have ranged from 19 to 74 years of age and have involved both men and women with varying degrees of AGA. Patients with scarring alopecia including FFA, LLP and CCCA were also treated and experienced marked improvement in hair growth and reduction of scarring. Over 95% of applicant's patients to date have experienced improved hair growth and decreased hair loss. Most patients have noticed a 30-40% improvement in the areas of hair loss on the scalp including the vertex of the scalp and the anterior/temporal areas which are often resistant to other topical treatments. Based on applicant's clinical observations, the younger age of the patient and the extent of hair loss involvement seems to play a role in their ability to respond to the inventive mixture. Without wishing to be bound by a theory, younger patients presumably have more active stem cells and anagen (growing phase) follicles and less telogen (resting phase) follicles, and appear to respond more quickly and with better hair growth than older more involved patients with AGA. Some of these patient's before and after treatment photos are attached to this disclosure as exhibits demonstrating the impressive and fast results from the applicant's novel formulation. On skilled in the art will also appreciate such respective before and after photos in Exhibits (2A, 2B, 3A, 3B, 4A, 4B, 5A and 5B). Individual results from the treatment may vary.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

What is claimed is:

1. A composition comprising, (a) and one or more of (b) and (c) as active ingredients in therapeutic amounts effective to promote hair growth of a subject:
(a) platelet rich plasma (“PRP”)
(b) dehydrated human amnion/chorion membrane allograft (“dHACM”);
(c) amnionic membrane allograft suspension (“AMA”);
wherein said therapeutic amounts of (b) or (c) when present is in the range of 25 mg to 100 mg.

2. The composition of claim 1, wherein said therapeutic amount of (a) is in the range of 12-18 ml.

3. The composition of claim 1, which further comprises an acceptable formulation for promoting hair growth of a subject.

4. The composition of claim 1, which is injectable.

5. The composition of claim 1, wherein the subject may have a hair loss disorder selected from one or more of the group consisting of alopecia, scarring alopecia, alopecia greata, androgenic alopecia, alopecia areata, alopecia universalis, involutional alopecia, trichotillomania, telogen effluvium, anagen effluvium, cicatricial, scalp thinning, hair shaft abnormalities, infectious hair disorders, genetic disorders, hair loss due to chemotherapy, hormonal imbalance, fungal infection, medication intake, and chemical hair treatment.

6. The composition of claim 1, wherein the subject is suffering from hair loss, said hair loss being associated with at least one disease selected from the group consisting of diabetes, lupus, poor nutrition, mental stress, and physical stress.

7. A kit comprising:
a first container containing a therapeutic amount of dehydrated human amnion/chorion membrane allograft (“dHACM”) and/or amnionic membrane allograft suspension (“AMA”); and
a second container for the collection of platelet rich plasma (“PRP”),
a plurality of syringes used for one or more of (i) the collection of PRP; (ii) mixing PRP with said first container of dHACM and/or AMA to form a therapeutic mixture, and (iii) injecting said therapeutic mixture intradermally or subcutaneously into a subject.
wherein each of the total amounts of the collected PRP, and the first container of dHACM and/or AMA is an amount effective to promote hair growth of a subject suffering from hair loss.
